(19) United States
(12) Patent Application Publication
(10) Pub. No.: US 2014/0378648 A1
(43) Pub. Date: Dec. 25, 2014

(54) METHOD FOR PREPARING A COMPOUND COMPRISING AT LEAST ONE BETA-HYDROXY-URETHANE UNIT AND/OR AT LEAST ONE UPSILON-HYDROXY-URETHANE UNIT

(71) Applicants: Aurélien Soules, Capestang (FR); Sylvain Caillol, Montpellier (FR); Jean-Paul Joubert, Montpellier (FR); José Martins, Frontignan (FR); Bernard Boutevin, Montpellier (FR)

(72) Inventors: Aurélien Soules, Capestang (FR); Sylvain Caillol, Montpellier (FR); Jean-Paul Joubert, Montpellier (FR); José Martins, Frontignan (FR); Bernard Boutevin, Montpellier (FR)

(21) Appl. No.: 14/354,574

(22) PCT Filed: Oct. 26, 2012
(86) PCT No.: PCT/FR2012/000437

§ 371 Date: Sep. 3, 2014

(30) Foreign Application Priority Data
Oct. 28, 2011 (FR) 1159818

(51) Int. Cl. C07C 269/04; C08G 71/04

(52) U.S. Cl. CPC C07C 269/04; C08G 71/04
USPC 528/371; 560/158

(57) ABSTRACT

The invention relates to a method for preparing a compound comprising at least one β-hydroxy-urethane unit and/or at least one γ-hydroxy-urethane unit, consisting in reacting a compound (A) comprising at least one cyclocarbonate reactive unit with a compound (B) comprising at least one amino reactive unit (—NH2) in the presence of a catalyst. The invention also relates to the use of a catalyst comprising at least one organometallic complex and a co-catalyst chosen from the group of Lewis bases and/or tetra-alkyl ammonium salts, in order to catalyse the method of the invention. The invention relates to the technical field of preparing urethane derivatives.

### TECHNICAL FIELD OF THE INVENTION

An object of the invention is a method for preparing a compound comprising at least one β-hydroxy urethane unit and/or at least one γ-hydroxy-urethane unit, comprising reacting a compound A comprising at least one cyclocarbonate reactive unit with a compound B comprising at least one amino reactive unit (—NH₂) in the presence of a catalyst. An object of it is also the use of a catalyst comprising at least one organometallic complex and a cocatalyst selected from the group of Lewis bases, and/or salts of tetra-alkyl ammonium, in order to catalyze the method according to the invention.

It relates to the field of preparation of urethane derivatives.

### PRIOR ART

Polyurethanes occupy a prominent place in the field of polymers, due both to their very diverse nature and their application in numerous products such as coatings, paints, adhesives, varnishes, foams, elastic fibers, insulating materials, dielectric resins, sheaths for electric cables as well as the medical devices.

Conventional polyurethanes are usually synthesized by polycondensation reaction between a di- or polyol and a di- or polyisocyanate. The toxicity and the instability of isocyanates constitute a major drawback. In addition, isocyanates are conventionally obtained from phosgene, itself a reactant hazardous to health and the environment.

Many research studies have been carried out to implement less harmful methods. Thus, for several years, a new method of preparation of polyurethanes has been considered. It appeared that the condensation reaction between a diamine and a molecule having two cyclocarbonate groups leads to the formation of polyurethanes, without using isocyanate compounds (Ochia, B, Satoh, Y, Endo, T, J, Polym Sci./Part A:. Polym Chem, 39, 4091, 2001). Such a method is described for example in the following patent documents: U.S. Pat. No. 5,340,889 (Crawford et al.), U.S. Pat. No. 2,802,022 (Groszos et al.), U.S. Pat. No. 4,758,615, U.S. Pat. No. 6,120,905 (Figovsky), U.S. Pat. No. 2,935,494 (Whelan et al.) U.S. Pat. No. 4,758,615 (Engel et al.), U.S. Pat. No. 3,072,613 (Whelan et al.), U.S. Pat. No. 3,072,613 (Whelan et al.), EP1070733 (Polymate LTD et al.). The advantage of this new method is that it leads to polyurethanes without isocyanates, having better hydrolytic stability than conventional polyurethanes because of the formation of a (—OH) hydroxyl group in the β position of the urethane (or carbamate) [Figovski 0., Improving the Protective Properties of Non-Metallic Corrosion Resistant Materials and Coatings. Medeleev Journal of Chemical Society, NY, USA 1988 Vol 33 No 3. pp. 31-36]. However, the activation energy of the reaction is very high. A quantitative conversion of reactants is achieved when the polycondensation is carried out at temperatures greater than 80° C. and when the reaction time is greater than 200 hours.

In order to optimize this condensation reaction, various methods advise the use of catalysts. Among these methods, those described in patent documents U.S. Pat. No. 5,055,542 (Honel et al.), U.S. Pat. No. 5,132,458 (Honel et al.), U.S. Pat. No. 5,977,262 (Anderson), WO2003066580 (UCB, S. A.), WO2005016993 (HUNTSMAN ADVANCED MATERIALS GMBH) U.S. Pat. No. 4,268,684 (Arthur G. et al.), EP0065026 (DOW CHEMICAL Co) can be cited. The catalysts used are generally selected from the metal salts, Lewis bases, alkali metal salts, quaternary ammonium salts, and modified clays. However the temperature and time of reaction are high, which is highly disadvantageous industrially.

There is therefore a need for an efficient method of synthesis of hydroxyurethanes, in particular compounds comprising at least one β-hydroxy-urethane unit and/or at least one γ-hydroxy-urethane unit.

Thus, a first object of the present invention is to provide a method of preparation of compounds comprising at least one β-hydroxy-urethane unit and/or at least one γ-hydroxy-urethane unit, which is significantly more efficient than the known methods of the prior art.

Another object of the invention is to provide a method for preparing compounds comprising at least one β-hydroxy-urethane unit and/or at least one γ-hydroxy-urethane unit, which can be produced with high efficiency, thus meeting the economic demands of the industry.

An object of the invention is also to provide a method for preparing compounds comprising at least one β-hydroxy-urethane unit and/or at least one γ-hydroxy-urethane unit, which can be produced in less stringent conditions than the methods heretofore known, particularly with regard to the reaction temperature.

An object of the invention is also to provide a method enabling leading, in a very short reaction time, to compounds comprising at least one β-hydroxy-urethane unit and/or at least one γ-hydroxy-urethane unit, with better quality than those obtained by the known methods of the prior art.

Still another object of the invention is to provide a method for preparing compounds comprising at least one β-hydroxy-urethane unit and/or at least one γ-hydroxy-urethane unit, which aims to better preserve the health of operators and better respect the environment.

### DISCLOSURE OF INVENTION

It has now been found that these goals can be achieved in whole or in part by the method described below.

The solution provided by the invention is a method for preparing a compound comprising at least one β-hydroxy urethane unit and/or at least one γ-hydroxy-urethane unit, comprising reacting a compound A comprising at least one cyclocarbonate reactive unit with a compound B comprising at least one amino reactive unit (—NH₂) in the presence of a catalyst. This method is remarkable in that the catalyst comprises at least one organometallic complex and a cocatalyst selected from the group of Lewis bases, and/or salts of tetra-alkyl ammonium.

Unexpectedly, the applicant has discovered that in its research using a catalyst comprising at least one complex combined with a organometallic cocatalyst, could remarkably improve the method of preparation of compounds comprising at least one β-hydroxy-urethane unit and/or at least one γ-hydroxy-urethane unit.

Indeed, the use of such a catalyst enables:
carrying out the method of the invention at temperatures lower than 170° C., usually below 100° C., preferably below 50° C., more preferentially at ambient temperature (approximately 25° C.). In particular, in some cases, especially when compound A and/or B compound is weakly reactive (e.g. 3-aminomethyl-3,5,5-trimethyl cyclohexylamine, the polyamine family masked polyketimines, or alternatives) or has an oligomeric or polymeric nature, the method of the invention can address the need for homogenization of the reaction mixture or masked amine release, to be conducted at temperatures above ambient temperature and less than 100° C., in particular at the ambient temperature and less than 170° C.
to increase the reaction kinetics significantly, and
to obtain compounds comprising at least one β-hydroxy-urethane unit and/or at least one γ-hydroxy-urethane unit with a very good yield and very good quality, and in a time much shorter than the catalysts promoted by the known methods, this reaction time being generally less than 600 minutes, usually less than 480 minutes, and in certain cases the reaction time can be only a few minutes or even seconds.

Moreover, such a method does not require the use of toxic chemicals such as isocyanates and can be implemented with little or no solvent where necessary.

The method according to the invention therefore has great potential.

Further preferred features of the invention are listed below, each of these characteristics can be considered alone or in combination with outstanding characteristics defined above:
the organometallic complex contains an alkali, alkaline earth, or transition metal selected from Groups IA, IIA, IIIA, IIIB, IVA, IVB, VB, VIIB, VIIB and VIII of the periodic table, and at least two β-diketonate ligands.
the organometallic complex is selected from the following list: tris(acetylacetonate) aluminum(III), tris(hexafluoroacetylacetonate) aluminum(III), tris(trifluoroacetylacetonate) aluminum), tris(2,2,6,6-tetramethyl-3,5-heptanedionato) aluminum(III), bis(acetylacetonate) calcium(II), tris(acetylacetonate) chromium(III), tris(2,2,6,6-tetramethyl-3,5-heptanedionate) chromium(III), tris(acetylacetonate) cobalt(III), tris(acetylacetonate nitro) cobalt(III), tris(2,2,6,6-tetramethyl-3,5-heptanedionate(III), bis(acetylacetonate) copper(III), bis(2,2,6,6-tetramethyl-3,5-heptanedionate) copper(II), tris(acetylacetonato) gallium (III), acetylacetonate hafnium(IV), tris(acetylacetonato) indium(III), tris(acetylacetonate) iron(III), tris(2,2,6,6-tetramethyl-3,5-heptanedionate) iron(III), tris(acetylacetonate) manganese(III), bis(acetylacetonate) nickel(III), bis(acetylacetonate) palladium (II), bis(trifluoroacetylacetonate) palladium (II), acetylacetonate sodium bis(acetylacetonate) titanium oxide(IV), bis(2,2,6,6-tetramethyl-3,5-heptanedionate) titanium oxide(IV), tris(2,2,6,6-tetramethyl-3,5-heptanedionato)-titanium), tris(2,2,6,6-tetramethyl-3,5-heptanedionato)oxotitanium(IV), dichloro-bis(2,2,6,6-tetramethyl-3,5-heptanedionato) titanium(IV), (diisopropoxide)bis(acetylacetonate) titanium(IV), di(isopropoxide)bis(2,2,6,6-tetramethyl-3,5-heptanedionato) titanium(IV), bis(acetylacetonate) zinc (II), tetrakis(acetylacetonate) zirconium(IV), tetrakis(hexafluoroacetylacetonate) zirconium(IV), tetrakis(2,2,6,6-tetramethyl-3,5-heptanedionato) zirconium(IV), tetrakis(trifluoroacetylacetonate) zirconium(IV).
the molar organometallic complex/compound A ratio is from 0.001 to 0.05, preferably between 0.002 and 0.01.
the catalyst further comprises a cocatalyst is selected from trialkyl amines, aromatic heterocyclic amines, trialkylphosphines, triarylphosphines, trialkyl phosphites, triaryl phosphites, tetralakyl ammonium salts, or mixtures thereof.
The cocatalyst is used at a proportion of 1.5 to 3 moles per mole of organometallic complex, preferably 2 moles per mole of organometallic complex.
compound A is selected from the group of the compounds, oligomers or polymers terminated with at least one cyclocarbonate, preferably at least two cyclocarbonates.
Compound A is a compound satisfying:
the general formula A1:

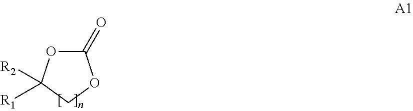

wherein:
n represents an integer of 1 or 2,
R₁ represents a hydrogen atom or a C₁₋₆ alkyl group, linear or branched, saturated or unsaturated, optionally substituted by one or multiple substituents selected from halogen, hydroxy, cyano, carboxy, trifluoromethyl, C₁₋₆ alkyl, C₁₋₆ alkylamino, C₁₋₆ dialkylamino, C₁₋₆ alkoxy, C₁₋₆ alkyl carboxylate,
R₂ represents a hydrogen atom or a group selected from: a C₁₋₂₀ alkyl, linear or branched, saturated or unsaturated, wherein non-neighboring methylene units can be replaced by radicals —O—, —S—, —S(O)—, —SO₂—, —O—C(═O)—, —N(R²¹)—, —N(R²¹)—C(═O)—, —N(R²¹)—C(═O)—O—, —N(R²¹)—C(═O)—N(R²²)—, with R²¹ and R²², being identical or different, representing hydrogen atoms or radicals selected from C₁₋₆ alkyl or C₆₋₁₄ aryl; C₃₋₁₀ cycloalkyl; C₆₋₁₄ aryl; C₃₋₁₀ heterocycle having one or multiple heteroatoms selected from N, O, S; C₄₋₁₃ heteroaryl having one or multiple heteroatoms selected from N, O, S; the members of this group being optionally substituted by one or multiple substituents selected from halogen, hydroxy, cyano, carboxy, trifluoromethyl, C₁₋₆ alkyl, C₁₋₆ alkylamino, C₁₋₆ dialkylamino, C₁₋₆ alkoxy, C₁₋₆ alkylcarboxylate,
or, the general formula A2:

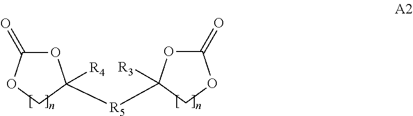

wherein:
n, n′, identical or different, represent an integer equal to 1 or 2,
R₃ and R₄, identical or different, represent a hydrogen atom or a C₁₋₆ alkyl group, linear or branched, saturated or unsaturated, optionally substituted by one or multiple substituents selected from halogen, hydroxy, cyano, carboxy, trifluoromethyl, C₁₋₆ alkylamino, C₁₋₆ dialkylamino, C₁₋₆ alkoxy, C₁₋₆ alkyl carboxylate,
R₅ represents:
a direct bond,
a bivalent group selected from: a C₁₋₂₀ alkylene, linear or branched, saturated or unsaturated, and wherein non-neighboring methylene units can be replaced by radicals —O—, —S—, —S(O)—, —SO₂—, —C(═O)—O—, —N(R²¹)—, —N(R²¹)—C(═O)—, —N(R²¹)—C(═O)—O—, —N(R²¹)—C(═O)—N(R²²)—, with R²¹ and R²² identical or different, representing hydrogen or selected from C₁₋₆ alkyl or C₆₋₁₄ aryl radicals; C₃₋₁₀ cycloalkyl; C₆₋₁₄ aryl; C₃₋₁₀ heterocyclic having one or multiple heteroatoms selected from N, O, S; C₄₋₁₃ heteroaryl having one or multiple heteroatoms selected from N, O, S; the members of this group being optionally substituted by one or multiple substituents selected from halogen, hydroxy, cyano, carboxy, trifluoromethyl, C₁₋₆ alkyl, C₁₋₆ alkylamino, C₁₋₆ dialkylamino, C₁₋₆ alkoxy, C₁₋₆ alkylcarboxylate,
or, a bivalent group having the following general formula I:

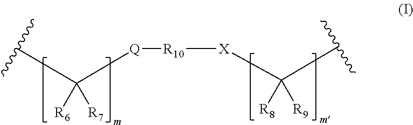

wherein:
m and m′, identical or different, designate an integer from 1 to 100,
R₆, R₇, R₈ and R₉, identical or different, represent hydrogen atoms or alkyl radicals, C₁₋₆ linear or branched, saturated or unsaturated, and optionally substituted by one or multiple substituents selected from halogen, hydroxy, cyano, carboxy, trifluoromethyl, C₁₋₆ alkyl, C₁₋₆ alkylamino, C₁₋₆ dialkylamino, C₁₋₆ alkoxy, C₁₋₆ alkyl carboxylate,
Q and X, identical or different, represent a group —O—C(═O)—, —N(R²¹)—, —N(R²¹)—C(═O)—, —N(R²¹)—C(═O)—O—, —N(R²¹)—C(═O)—N(R²²)—, with R²¹ and R²² as defined above,]
R₁₀ represents a bivalent group selected from a C₁₋₂₀ alkylene, linear or branched, saturated or unsaturated, and wherein non-neighboring methylene units can be replaced by radicals —O—, —S—, —S(O)—, —SO₂—, —C(═O)—O—, —N(R²¹)—, —N(R²¹)—C(═O)—, —N(R²¹)—C(═O)—O—, —N(R²¹)—C(═O)—N(R²²)—, with R²¹ and R²² are as defined above; a C₃₋₁₀ cycloalkyl; C₆₋₁₄ aryl; C₃₋₁₀ heterocyclic having one or multiple heteroatoms selected from N, O, S; C₄₋₁₃ heteroaryl having one or multiple heteroatoms selected from N, O, S; the members of this bivalent group being optionally substituted by one or multiple substituents selected from halogen, hydroxy, cyano, carboxy, trifluoromethyl, C₁₋₆ alkyl, C₁₋₆ alkylamino, C₁₋₆ dialkylamino, C₁₋₆ alkoxy, C₁₋₆ alkylcarboxylate-alkyl;
or a group satisfying one of the formulas II or III:

—(CH₂—CH₂—O)ₜ—CH₂—CH₂— (II), or

C(═O)—CH₂—O—(CH₂—CH₂—O)_{t′}—CH₂—C(═O)— (III)

wherein:

t, t′, identical or different, designate a whole number from 1 to 100.
compound A is a compound A3 obtained by:
the reaction of the 4-hydroxymethyl-1,3-dioxolan-2-one with a compound comprising one or multiple isocyanate functions,
the esterification reaction of 4-hydroxymethyl-1,3-dioxolan-2-one with a compound comprising one or multiple carboxylic acid functions,
the reaction of the triglycidyl isocyanurate with carbon dioxide, or the reaction of a compound comprising two or several glycidyl ether groups with carbon dioxide.
compound B is selected from the group of compounds, oligomers or polymers terminated with at least one reactive unit (—NH₂), preferably by at least two reactive units (—NH₂).
the one or multiple reactive units (—NH₂) are carried by saturated carbons (sp3), which are also carriers of at least one hydrogen, preferably two hydrogens.
compound B comprising at least one amino reactive unit (—NH₂) is selected from the following list: butylamine; hexylamine; cyclohexylamine; ethanolamine; propanolamine; ethylene diamine; propylene diamine; butylene diamine; pentamethylene diamine; hexamethylene diamine; heptamethylene diamine; tetramethylene diamine; octamethylene diamine; nonamethylene diamine; decamethylene diamine; 2-methyl penta-methylene diamine; undecamethylene diamine; dodecamethylene diamine; xylylene diamine; isophorone diamine; trimethyl hexamethylene diamine; 1,2-diaminocyclohexane; 1,4-diaminocyclohexane; 4,4′-diaminocyclohexyl methane; 2-(2-aminoethoxy) ethanol; bis-(3-methyl-4-aminocyclohexyl) methane; 2,2-bis(4-aminocyclohexyl) propane; nitrile tri(éthaneamine); bis-(3-aminopropyl)methylamine; 3-amino-1-(methylamino) propane; 3-amino-1-(cyclohexylamino) propane; N-(2-hydroxyethyl)ethylene diamine; 4,7-dioxadecane-1,10-diamine; 4,9-dioxadodecane-1,12-diamine; 7-methyl 4,10-dioxamidécane-1,13-diamine; a polyether monoamine preferably selected from amino hydroxy polyethylene glycol and methoxypolyethylene glycol amine, a polyether diamine, preferably selected from a polyoxyethylene diamine and a polyoxypropylene diamine; and/or an oligomer or polymer terminated by at least one amino reactive unit (—NH₂), preferably by two or several amino reactive units, said oligomer or polymer capable of having a polyamide, polyether and/or polyester skeleton.
compound A and compound B are mixed so that the molar ratio of compound B over compound A is between 0.5 and 2, preferably equal to 1.
compound A and compound B are pre-mixed with a plasticizer before contacting with the catalyst comprising at least one organometallic complex, optionally in combination with a cocatalyst.
The method is operated at a temperature between 0° C. and 170° C., preferably 0° C. and 100° C., more preferentially at ambient temperature.

Another aspect of the invention relates to the use of at least one organometallic complex containing an alkali, alkaline earth, or transition metal selected from Groups IA, IIA, IIIA; IIIB, IVA, IVB, VB, VIIB, VIIB and VIII of the periodic table, and at least two acetylacetonato ligands, and optionally associated with a co-catalyst, to catalyze the condensation reaction between a compound A comprising at least one cyclocarbonate reactive unit and a compound B comprising at least one amino reactive unit (—NH₂).

### DESCRIPTION OF THE FIGURES

Other advantages and features of the invention will become better apparent upon reading the description of a preferred implementation mode which will follow, with reference to the attached figures, made by way of indicative and non-limiting examples and in which:

FIG. 1 shows an FTIR spectrum made at 25 seconds of the crude reactive of the condensation reaction between trimethylhexamethylenediamine and 4-(hydroxymethyl)-1,3-dioxolan-2-one in the presence of a catalytic amount of triethylamine;

FIG. 2 shows monitoring, by FTIR spectroscopy, of the progression of the condensation reaction between trimethylhexamethylenediamine and 4-(hydroxymethyl)-1,3-dioxolan-2-one catalyzed by a catalyst according to the invention or by certain catalysts advocated in known prior art methods;

FIG. 3 shows monitoring, by FTIR spectroscopy, of the progression of the condensation reaction between the diamine and trimethylhexamethylene acetate(2-oxo-1,3-dioxolan-4-yl) methyl catalyzed by the catalyst comprising tris(2,4-pentanedione) chromium (III) combined with triethylamine. (Overlay of FTIR spectra made on samples of the reaction mixture at 25 seconds and 180 seconds).

FIG. 4 shows monitoring, by FTIR spectroscopy, the progression of the condensation reaction between tetraethylenepentamine and 4-(hydroxymethyl)-1,3-dioxolan-2-one catalyzed, at ambient temperature, by the catalyst comprising tris(2,4-pentanedione) chromium (III) combined with triethylamine.

FIG. 5 shows monitoring, by FTIR spectroscopy, of the progression of the condensation reaction between the 3-aminomethyl-3,5,5-trimethylcyclohexylamine and 4-(hydroxymethyl)-1,3-dioxolan-2-one catalyzed, at ambient temperature, by the catalyst comprising tris(2,4-pentanedione) chromium (III) combined with triethylamine.

FIG. 6 shows monitoring, by FTIR spectroscopy, of the progression of the condensation reaction between Jeffamine® D230 and 4-(hydroxymethyl)-1,3-dioxolan-2-one catalyzed, at ambient temperature, by the catalyst comprising tris(2,4-pentanedione) chromium (III) combined with triethylamine.

FIG. 7 shows monitoring, by FTIR spectroscopy, of the progression of the condensation reaction between tetraethylenepentamine and a compound comprising two cyclocarbonate reactive units obtained by the reaction of 4-(hydroxymethyl)-1,3-dioxolan-2-one with the diisocyanate Desmodur N3400, the condensation reaction being catalyzed, at ambient temperature, by the catalyst comprising tris(2,4-pentanedione) chromium (III) combined with triethylamine.

### IMPLEMENTATION MODES OF THE INVENTION

In the context of the present invention, unless otherwise stated in the text, it is meant:
“halogen atom”: a fluorine, a chlorine, a bromine or a iodine, preferably a fluorine atom;
“alkyl”: a saturated or unsaturated, linear or branched, aliphatic group, having 1 to 20 successive carbon atoms (abbreviated as C₁₋₂₀ alkyl). Methyl, ethyl, propyl, isopropyl, butyl, isobutyl, tertbutyl, pentyl, hexyl, heptyl, octyl, decyl, undecyl, dodecyl, tridecyl, tetradecyl, pentadecyl, hexadecyl, heptadecyl, octadecyl, nonadecyl, icosanyle, propenyl, butenyl, pentenyl, 1-hexenyl, heptenyl, octenyl, nonenyl, decenyl, undecenyl, oleyl, and linolenyl linolyle etc. can be cited as examples. Except in the case where it is specified, the alkyl group can comprise 1 to 6 successive carbon atoms (abbreviated as C₁₋₆ alkyl). The alkyl groups can be optionally substituted by an aryl group such as defined below, in which case it is called arylalkyl group. Examples of arylalkyl groups are notably benzyl, phenethyl, phenylpropyl, etc.
“alkylene”: an alkyl group such as defined above, divalent.
“cycloalkyl”: a monovalent cyclic alkyl group having 3 to 10 carbon atoms, saturated or unsaturated. The groups cyclopropyl, methylcyclopropyl, cyclobutyl, cyclopentyl, cyclohexyl, cyclooctyl, cyclononyl, cyclodecyl, etc. can be cited as examples.
“cycloalkylene”: a cycloalkyl group such as defined above, divalent.
“aryl”: an aromatic hydrocarbon group having 6 to 14 carbon atoms (abbreviated by C₃₋₁₄ aryl), yet more preferentially 6 carbon atoms. The aryl groups noteably include the radicals phenyl, naphthyl and biphenyl.
“arylene”: an aryl group such as defined above, divalent.
“heterocycle”: a saturated or unsaturated mono- or bi-cyclic group (nonaromatic) optionally fused or bridged with 3 to 10 carbon atoms (abbreviated by C₃₋₁₀ heterocyclic) of which at least one atom is selected among atoms of oxygen, nitrogen or sulfur. The groups 2,3-dihydrobenzofuran and 1,4-benzodioxane, phthalimide, etc. will be cited for example.
“heterocyclene”: a heterocyclyl group such as defined above, divalent.
“heteroaryl”: an aromatic mono- or bi-cyclic group, comprising between 4 and 13 carbon atoms and comprising between 1 and 3 heteroatoms, such as nitrogen, oxygen or sulfur. The groups furan, pyridine, thiazole, imidazole, benzothiophene etc. can be cited as examples of heteroaryl groups.
“heteroarylene”: a heteroaryl group such as defined above, divalent.
“alkoxy”: an —O-alkyl group where the alkyl group is such as defined above and has from 1 to 6 carbon atoms (abbreviated C₁₋₆ alkoxy).
“alkylamino”: an —NH-alkyl group where the alkyl group is as such as defined above and has from 1 to 6 carbon atoms (abbreviated by C₁₋₆ alkylamino).
“dialkylamino”: a group-N(alkyl)₂ where alkyl is as defined above and has from 1 to 6 carbon atoms (abbreviated as C₁₋₆ dialkylamino).
“alkylcarboxylate”: a —O—C(═O)-alkyl group where the alkyl group is such as defined above and has from 1 to 6 carbon atoms (abbreviated by C₁₋₆ alkyl carboxylate).

The alkyl, alkylene, cycloalkyl, cycloalkylene, aryl, arylene, heterocyclic, heterocyclene, heteroaryl and heteroarylene groups can, if required, be substituted by one or multiple substituents selected among halogen, hydroxy, cyano, carboxy, trifluoromethyl, C₁₋₆ alkyl, C₁₋₆ alkylamino, C₁₋₆ alkyl, C₁₋₆ dialkylamino, C₁₋₆ alkoxy, C₁₋₆ alkyl carboxylate.
“compound comprising at least one β-hydroxy-urethane unit and/or at least one γ-hydroxy-urethane unit”: a compound comprising one or multiple β-hydroxy-urethane units; one or multiple γ-hydroxy-urethane units; or one or multiple β-hydroxy-urethane units and one or multiple γ-hydroxy-urethane units.

The invention is based on the discovery that the presence of a catalyst based on organometallic complexes, optionally combined with a co-catalyst, enables the acceleration of the formation of compounds comprising at least one β-hydroxy-urethane unit and/or at least one γ-hydroxy-urethane under mild conditions and with an excellent conversion rate.

The organometallic complexes suitable for the implementation of the method of the invention is one containing an alkali, alkaline earth or transition metal selected among the Groups IA, IIA, IIIA; IIIB, IVA, IVB, VB, VIIB, VIIB and VIII of the periodic table, and at least two 1-diketonate ligands.

As examples of transition metal involved in the organometallic complex, antimony, silver, cadmium, chromium, cobalt, copper, tin, iron, gallium, germanium, hafnium, indium, iridium, manganese, mercury, molybdenum, nickel, niobium, gold, osmium, palladium, platinum, rhenium, rhodium, ruthenium, scandium, tantalum, titanium, technetium, tungsten, vanadium, yttrium, zinc, and zirconium can be cited.

The β-diketonate ligands in the organometallic complex are preferably selected from the acetylacetonato, 1,1,1,5,5,5-hexa-fluoro-acetyl, 1,1,1-trifluoro-acetylacetonate, 1,1,1-trifluoro-5,5-di-méthylacétylacétonate and 2,2,6,6-tetramethyl-3,5-heptanedionate.

Preferably, the organometallic complex is selected from the following list: tris(acetylacetonate) aluminum(III), tris(hexafluoroacetylacetonate) aluminum(III), tris(trifluoroacetylacetonate) aluminum(III), tris(2,2,6,6-tetramethyl-3,5-heptanedionato) aluminum(III), bis(acetylacetonate) calcium(II), tris(acetylacetonate) chromium(III), tris(2,2,6,6-tetramethyl-3,5-heptanedionate) chromium(III), tris(acetylacetonate) cobalt(III), tris(acetylacetonate nitro) cobalt(III), tris(2,2,6,6-tetramethyl-3,5-heptanedionate) cobalt(III), bis(acetylacetonate) copper(II), bis(2,2,6,6-tetramethyl-3,5-heptanedionate) copper(II), tris(acetylacetonato) gallium (III), acetylacetonate hafnium(IV), tris(acetylacetonato) indium(III), tris(acetylacetonate) iron(III), tris(2,2,6,6-tetramethyl-3,5-heptanedionate) iron(III), tris(acetylacetonate) manganese(III), bis(acetylacetonate) nickel(III), bis(acetylacetonate) palladium (II), bis(trifluoroacetylacetonate) palladium (II), acetylacetonate sodium bis(acetylacetonate) titanium oxide(IV), bis(2,2,6,6-tetramethyl-3,5-heptanedionate) titanium oxide(IV), tris(2,2,6,6-tetramethyl-3,5-heptanedionato)-titanium(III), tris(2,2,6,6-tetramethyl-3,5-heptanedionato)oxotitanium(IV), dichloro-bis(2,2,6,6-tetramethyl-3,5-heptanedionato) titanium(IV), (diisopropoxide)bis(acetylacetonate) titanium(IV), di(isopropoxide)bis(2,2,6,6-tetramethyl-3,5-heptanedionato) titanium(IV), bis(acetylacetonate) zinc (II), tetrakis(acetylacetonate) zirconium(IV), tetrakis(hexafluoroacetylacetonate) zirconium(IV), tetrakis(2,2,6,6-tetramethyl-3,5-heptanedionato) zirconium(IV), tetrakis(trifluoroacetylacetonate) zirconium(IV).

These organometallic complexes are commercially available or can be prepared by methods known to those skilled in the art for example by the reaction between a transition metal salt with acetylacetone in the presence of a base as described for example by RC Mehrotra in “Metal Beta-diketonates and Allied Derivatives” Academic Press, 1978 or by Fackler, J P, Jr., “Metal Complexes β-Ketoenolate” in Progress in Inorganic Chemistry, FA Cotton, Ed, Interscience: New York, 1966, Vol. 7, p. 471.

Bis(acetylacetonate) calcium (II), tris(acetylacetonate) chromium (III), tris(acetylacetonate) iron (III), tris(acetylacetonate) cobalt (III), bis(acetylacetonate) zinc (II), le tris(acétylacétonate) aluminum (III), and bis(acetylacetonate) nickel(II), represent the organometallic complexes particularly well suited for the implementation of the method according to the invention, in particular for reason of their low cost.

Of course mixtures of multiple organometallic complexes can be used.

The amount of organometallic complex used can vary within wide limits. It is generally such that the organometallic complex/compound molar ratio is between 0.001 and 0.05, preferably between 0.002 and 0.01.

According to a particularly preferred variant of the invention, the organometallic complex is combined with a co-catalyst which. The Applicant has in fact found that the presence of such a co-catalyst was enabling further acceleration of the formation of compounds comprising at least one β-hydroxy-urethane unit and/or at least one γ-hydroxy-urethane unit. Such a co-catalyst can be preferably selected from the group consisting of Lewis bases, tetraalkylammonium salts, and their mixture.

As non-limiting examples of such Lewis base one can cite trialkylamines such as triethylamine, tributylamine, or 1,4-diazabicyclooctane; tetralakyl ammonium salts such as tetrabutylammonium bromide; aromatic heterocyclic amines such as pyridine, 4-dimethylaminopyridine, or N-methyl imidazole; the trialkylp osp ines such as osphine triéthylp or butylphosphine; triarylphosphines such as triphenylphosphine; trialkyl phosphites such as trimethylphosphite, triethylphosphite, triisopropylphosphite, tri-n-butylphosphite; or triaryl phosphites such as triphenyl phosphite. Among the Lewis base, triethylamine, tributylamine or 1,4-diazabicyclooctane are particularly preferred.

Examples of quaternary ammonium salts include tetrabutylammonium bromide and tetrabutylammonium chloride.

Of course mixtures of multiple co-catalysts can be used.

The cocatalyst is generally used at a proportion of 0.5 to 3 moles per mole of organometallic complex, preferably 1.5 to 3 moles per mole of organometallic complex, preferentially at a proportion of 2 moles per mole of organometallic complex.

According to a preferred implementation mode, the method according to the invention is particularly suitable for the preparation of a compound comprising at least one β-hydroxy-urethane unit and/or at least one γ-hydroxy-urethane unit from a compound A comprising at least one cyclocarbonate reactive unit and a compound B comprising at least one amino reactive unit (—NH₂).

A compound suitable for carrying out the method of the invention can be selected from the group of compounds, oligomers or polymers terminated with at least one cyclocarbonate reactant unit, preferably at least two cyclocarbonate reactive units, these oligomers or polymers can be linear or branched.

Thus, according to a first variant, compound A is a compound satisfying the general formula A1:

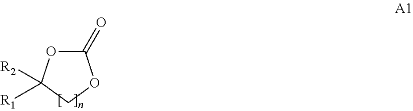

wherein
n represents an integer of 1 or 2,
R₁ represents a hydrogen atom or an C₁₋₆ alkyl group linear or branched, saturated or unsaturated and optionally substituted by one or multiple substituents selected from halogen, hydroxy, cyano, carboxy, trifluoromethyl, C₁₋₆ alkyl, C₁₋₆ alkylamino, C₁₋₆ dialkylamino, C₁₋₆ alkoxy, C₁₋₆ alkyl carboxylate,
R₂ represents a hydrogen atom or a group selected from a C₁₋₂₀, linear or branched, saturated or unsaturated, and wherein non-neighboring methylene units can be replaced by radicals —O—, —S—, —S(O)—, —SO₂—, —O—C(═O)—, —N(R²¹)—, —N(R²¹)—C(═O)—, —N(R²¹)—C(═O)—O—, —N(R²¹)—C(═O)—N(R²²)—, with R²¹ and R²² identical or different, representing hydrogen atoms or radicals selected among C₁₋₆ alkyl or C₆₋₁₄ aryl; a C₃₋₁₀ cycloalkyl; a C₆₋₁₄ aryl; a C₃₋₁₀ heterocycle having one or multiple heteroatoms selected from N, O, S; a C₄₋₁₃ heteroaryl having one or multiple heteroatoms selected from N, O, S; the members of this group being optionally substituted by one or multiple substituents selected from halogen, hydroxy, cyano, carboxy, trifluoromethyl, C₁₋₆ alkyl, C₁₋₆ alkylamino, C₁₋₆ dialkylamino, C₁₋₆ alkoxy, C₁₋₆ alkyl carboxylate.

By way of non-limiting example of compounds A1 used in the method of the invention include 1,3-dioxolan-2-one; 4-methyl-1,3-dioxolan-2-one; 4-ethyl-1,3-dioxolan-2-one; 4-(trifluoromethyl)-1,3-dioxolan-2-one; dimethyl-1,3-dioxolan-2-one; 4-ethenyl-1,3-dioxolan-2-one; 4-phenyl-1,3-dioxolan-2-one; 4-methyl-1,3-dioxan-2-one; 1,3-dioxan-2-one; 4-hydroxymethyl-1,3-dioxolan-2-one; acetate(2-oxo-1,3-dioxolan-4-yl) methyl; 4-(methoxymethyl)-1,3-dioxolan-2-one; 4-(propan-2-yloxy)-1,3-dioxolan-2-one; 4-(butoxymethyl)-1,3-dioxolan-2-one; 4-(naphthalen-1-yloxymethyl)-1,3-dioxolan-2-one; 4-(phénylméthoxyméthyl)-1,3-dioxolan-2-one; 4-(hexadécoxyméthyl)-1,3-dioxolan-2-one; 4-[(E)-octadec-9-enoxy]-1,3-dioxolan-2-one; 4-(octoxyméthyl)-1,3-dioxolan-2-one; 4-(1-hydroxy-2-phenylethyl)-1,3-dioxolan-2-one; 4-(1-hydroxy-2-phénylméthoxyéthyl)-1,3-dioxolan-2-one; 4-[[4-(6-methoxy-2-phenyl-3,4-dihydronaphthalen-1-yl)phenoxy]methyl]-1,3-dioxolan-2-one; 4-(1-hydroxy-2-phénylméthoxyéthyl)-1,3-dioxolan-2-one.

According to another variant, compound A is a compound satisfying the general formula A2:

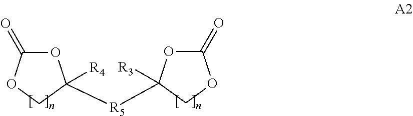

wherein
n, n′, identical or different, represent an integer equal to 1 or 2,
R₃ and R₄, identical or different, represent a hydrogen atom or C₁₋₆ alkyl group, linear or branched, saturated or unsaturated, and optionally substituted by one or multiple substituents selected from halogen, hydroxy, cyano, carboxy, trifluoromethyl, C₁₋₆ alkyl, C₁₋₆ alkylamino, C₁₋₆ dialkylamino, C₁₋₆ alkoxy, C₁₋₆ alkyl carboxylate,
R₅ represents:
a direct bond,
a bivalent group selected from: a C₁₋₂₀ alkylene, linear or branched, saturated or unsaturated, and wherein non-neighboring methylene units can be replaced by radicals —O—, —S—, —S(O)—, —SO₂—, —C(═O)—O—, —N(R²¹)—, —N(R²¹)—C(═O)—, —N(R²¹)—C(═O)—O—, —N(R²¹)—C(═O)—N(R²²)—, with R²¹ and R²² identical or different, representing hydrogen atoms or radicals selected from C₁₋₆ alkyl or C₆₋₁₄ aryl; a C₃₋₁₀ cycloalkyl; a C₆₋₁₄ aryl; a C₃₋₁₀ heterocyclic having one or multiple heteroatoms selected from N, O, S; a C₄₋₁₃ heteroaryl having one or multiple heteroatoms selected from N, O, S; the members of this group being optionally substituted by one or multiple substituents selected from halogen, hydroxy, cyano, carboxy, trifluoromethyl, C₁₋₆ alkyl, C₁₋₆ alkylamino, C₁₋₆ dialkylamino, C₁₋₆ alkoxy, C₁₋₆ alkylcarboxylate,
or, a bivalent group having the following general formula I:

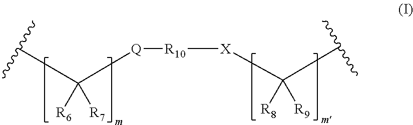

wherein:
m and m′, identical or different, designate a whole number from 1 to 100,
R₆, R₇, R₈ and R₉, identical or different, represent hydrogen atoms or alkyl radicals, C₁₋₆ linear or branched, saturated or unsaturated, and optionally substituted by one or multiple substituents selected from halogen, hydroxy, cyano, carboxy, trifluoromethyl, C₁₋₆ alkyl, C₁₋₆ alkylamino, C₁₋₆ dialkylamino, C₁₋₆ alkoxy, C₁₋₆ alkyl carboxylate,
Q and X, identical or different, represent a group —O—C(═O)—, —N(R²¹)—, —N(R²¹)—C(═O)—, —N(R²¹)—C(═O)—O—, —N(R²¹)—C(═O)—N(R²²)—, with R²¹ and R²² are as defined above,
R₁₀ represents a bivalent group selected from: a C₁₋₂₀ alkylene, linear or branched, saturated or unsaturated, and wherein non-neighboring methylene units can be replaced by radicals —O—, —S—, —S(O)—, —SO₂—, —C(═O)—O—, —N(R²¹)—, —N(R²¹)—C(═O)—, —N(R²¹)—C(═O)—O—, —N(R²¹)—C(═O)—N(R²²)—, with R²¹ and R²² identical or different, representing hydrogen atoms or radicals selected from C₁₋₆ alkyl or C₆₋₁₄ aryl; a C₃₋₁₀ cycloalkyl; a C₆₋₁₄ aryl; a C₃₋₁₀ heterocyclic having one or multiple heteroatoms selected from N, O, S; a C₄₋₁₃ heteroaryl having one or multiple heteroatoms selected from N, O, S; the members of this group being bivalent, optionally substituted by one or multiple substituents selected from halogen, hydroxy, cyano, carboxy, trifluoromethyl, C₁₋₆ alkyl, C₁₋₆ alkylamino, C₁₋₆ dialkylamino, C₁₋₆ alkoxy, C₁₋₆ alkylcarboxylate.

or, a group satisfying one of the formulas II or III:

—(CH₂—CH₂—O)ₜ—CH₂—CH₂— (II), or

C(═O)—CH₂—O—(CH₂—CH₂—O)_{t′}—CH₂—C(═O)— (III)

wherein:
t, t′, identical or different, designate a whole number from 1 to 100.

By way of non-limiting example of compounds satisfying the formula A2, 4-(2-oxo-1,3-dioxolan-4-yl)-1,3-dioxolan-2-one; bis[(2-oxo-1,3-dioxolan-4-yl)methyl]hexanedioate and 2,5-dithiahexane-1,6-diyl-4,4′-bis(1,3-dioxolan-2-one) can be cited.

According to yet another variant, compound A is a compound A3 obtainable by the condensation reaction of 4-hydroxymethyl-1,3-dioxolan-2-one with a compound comprising two or several isocyanate functions, such as methylene diphenyl diisocyanate; 1,3-diisocyanatobenzene; 1,4-diisocyanatobenzene; 2,4-diisocyanato-1-methylbenzene; 1-isocyanato-4-[(4-isocyanatocyclohexyl)methyl]-cyclohexane; 5-diisocyanatonaphthalene; 1,3-diisocyanato-2-methylbenzene; 1,6-diisocyanatohexane; 1-isocyanato-4-(4-isocyanato-3-methylphenyl)-2-methylbenzene; 1-isocyanato-4-(4-isocyanato-3-methoxyphenyl)-2-methoxybenzene; 1,3-bis(isocyanatomethyl)benzene; 1-isocyanato-4-(isocyanatomethyl)benzene; 5-isocyanato-1-(isocyanatomethyl)-1,3,3-trimethylcyclohexane; 1,4-diisocyanatocyclohexane; 2-[2-[[3-(isocyanatomethyl)phenyl]carbamoyloxy]ethoxy]-ethyl N-[3-(isocyanatomethyl) phenyl]carbamate; 1,3-diisocyanato-5-[(2-isocyanatophenyl)methyl]-2-methylbenzene; 1,3-diisocyanato-5-[(2-isocyanatophenyl)methyl]-2-methylbenzene; 1,10-diisocyanatodécane; 1,6-diisocyanato-2,3,4-trimethylhexane; 1,5-diisocyanato-2,2-dimethylpentane; 1,8-diisocyanatooctane; 1,4-diisocyanatobutane; 1,4-bis(isocyanatomethyl)cyclohexane; 3-[[3-(isocyanatomethyl)-phenyl]carbamoyloxy]butyl N-[3-(isocyanatomethyl)phenyl]carbamate; 1,3-bis[(5-isocyanato-1,3,3-trimethylcyclohexyl)methyl]urea; 1,6,11-undecane triisocyanate; ethyl ester L-lysine triisocyanate; triphenyl methane triisocyanate; 1,3-bis(6-isocyanatohexyl)-1-(6-isocyanatohexylcarbamoyl)urea; 1,3-diisocyanato-5-[(4-isocyanatophenyl)methyl]-2-methylbenzene; methyl-6-[2,2-bis[(5-isocyanato-6-methoxy-6-oxohexyl) carbamoyloxyméthyl]butoxycarbonylamino]-2-isocyanatohexanoate, or mixtures thereof.

Compound A3 can also be obtained by the esterification reaction of 4-hydroxymethyl-1,3-dioxolan-2-one with a compound comprising two or several functional carboxylic acids such as aliphatic dibasic acids or aromatic diacids. As examples of aliphatic diacids, ethanedioic acid; 1,3-propanedioic acid; 4-butanedioic acid; 5-pentanedioic acid; 1,6-hexanedioic acid; 1,7-heptanedioic acid; 1,8-octanedioic acid; 1,9-nonanedioic acid; 1,10-decanedioic acid; 1,11-undecanedioic acid or 1,12-dodecanedioic acid can be cited. As examples of aromatic diacids, benzene-1,2-dicarboxylic acid; benzene-1,3-dicarboxylic acid or benzene-1,4-dicarboxylic acid can be cited.

Compound A3 can also be obtained by the condensation reaction of triglycidyl isocyanurate (PolyScience Inc.) with carbon dioxide.

Compound A3 can also be obtained by reacting a compound having two or several glycidyl ether groups with carbon dioxide in the presence of a catalyst as described in patent documents U.S. Pat. No. 5,340,889 (Crawford et al.), U.S. Pat. No. 7,232,877 (Figovsky et al.), U.S. Pat. No. 5,175,231 (Rappoport et al.), U.S. Pat. No. 6,495,637 (Rappoport et al.). As examples of compounds comprising two or several glycidyl ether groups one can cite 2-[2-(oxiran-2-ylmethoxy)ethoxymethyl]oxirane (651 Quetol; Polyscience Inc.); 2-[3-(oxiran-2-ylmethoxy)-propoxymethyl]oxirane (PolyScience Inc.), 2-[4-(oxiran-2-ylmethoxy)-butoxymethyl]oxirane (PolyScience Inc.); 2-[2,2-bis(oxirane-2-ylméthoxyméthyl)butoxymethyl]oxirane (trimethylolpropane triglycidyl ether); 2-[[2-methyl-3-(oxiran-2-ylmethoxy)-2-(oxiran-2-ylméthoxyméthyl)propoxy]methyl]oxirane; 2-[[3-(oxiran-2-ylmethoxy)-1-(oxiran-2-ylméthoxyméthyl) propoxy]methyl]oxirane; 2-[[3-(oxiran-2-ylmethoxy)-2,2-bis(oxiran-2-ylmethoxymethyl)propoxy]methyl]oxirane (or pentaerythritol glycidyl ether); N,N-diglycidyW-glycidyloxyaniline (Aldrich®); a di- or polyglycidyl ether of HELOXY® series (HEXION® Specialty Chemicals); bis(polyoxyethylene bis[glycidyl ether]) (Sigma-Aldrich Co.); poly(propylene glycol) (600) bis[glycidyl ether](PolyScience Inc.); or selected from those described in patent U.S. Pat. No. 6,410,127 (Toray Industries, Inc.), epoxy resin or a bisphenol epoxy resin selected from those sold by the Dow Company (bisphenol A epoxy resin, bisphenol F epoxy resin, epoxy resin bisphenol A/F; modified bisphenol A epoxy resin, modified epoxy resin bisphenol A/F).

One can, of course, use mixtures of compounds comprising two or several cyclocarbonate reactive units, for example for the preparation of hydroxylated polyurethanes.

Compound B useful in the method of the invention can be selected from the group of compounds, oligomers or polymers terminated with at least one reactive unit (—NH₂), preferably by at least two reactive groups (—NH₂), these oligomers or polymers capable of being linear or branched.

Advantageously, the one or multiple reactive units (—NH₂) are carried by saturated carbons (sp3), which are also carriers of at least one hydrogen, preferably two hydrogens. As examples of compound B suitable for the implementation of the method according to the invention, one can cite: butylamine; hexylamine; cyclohexylamine; ethanolamine; propanolamine; ethylene diamine; propylene diamine (or 1,4-diaminopropane or trimethylene diamine); butylene diamine(1,4-diaminobutane or tetramethylene diamine); pentamethylene diamine; hexamethylene diamine; heptamethylene diamine; tetramethylene diamine; octamethylene diamine; nonamethylene diamine; decamethylene diamine; 2-methyl-pentamethylene diamine; undecamethylene diamine; dodecamethylene diamine; xylylene diamine; isophorone diamine; trimethyl hexamethylene diamine(2,2,4-trimethyl hexamethylenediamine and/or 2,4,4-trimethyl hexamethylene diamine or Vestamin® TMD); 1,2-diaminocyclohexane; 1,4-diaminocyclohexane; 4,4′-diaminocyclohexyl methane (1,4-bis(aminocyclohexyl)methane); 2-(2-aminoethoxy)ethanol; bis-(3-methyl-4-aminocyclohexyl)methane; 2,2-bis(4-aminocyclohexyl)propane; nitrile tris(éthaneamine); bis-(3-aminopropyl)methylamine; 3-amino-1-(methylamino) propane; 3-amino-1-(cyclohexylamino)propane; N-(2-hydroxyethyl)ethylene diamine; 4,7-dioxadecane-1,10-diamine; 4,9-dioxadodecane-1,12-diamine; 7-methyl-4,10-dioxamidécane-1,13-diamine; a polyether monoamine preferably selected from amino hydroxy polyethylene glycols and methoxypolyethylene glycol amines of the Jeffamine ® M series (Example M-600 (XTJ-505), M-1000 (XTJ-506), M-2005, M-2070); a polyether diamine, preferably selected from a polyoxyethylene diamine and a polyoxypropylene diamine of the Jeffamine® D series, ED and/or EDR (example JEFFAMINE® D-230, D-400 or D-2000; EDR-148 (XTJ-504), EDR-176 (XTJ-590), HK-511, ED-600 (XTJ-500), ED-900 (XTJ-501), ED-2003 (XTJ-502).

Compound B usable in the method of the invention can also be selected from triamines, tetramines, polyoxyethylene triamines selected from the JEFFAMINE® T series (eg, T-403, T-3000 (XTJ-509), T-5000) and the oligomers or polymers terminated by at least one amino reactive unit (—NH₂), preferably by two or several amino reactive units (NH₂), said oligomers or polymers capable of having a polyamide, polyether, and/or polyester skeleton. (Example: a polyamido polyamine such as those prepared by the method described in patent U.S. Pat. No. 5,391,826 (Speranza et al.)].

In general, compound A, comprising at least one cyclocarbonate reactive unit, and compound B, comprising at least one amino reactive unit (—NH₂), are mixed so that the molar ratio of compound B over compound A is between 0.5 and 2, preferably equal to 1. The molar ratio can vary around the stoichiometric ratio according to the desired terminal group in the final product.

The presence of a solvent is not necessary for carrying out the method according to the invention. However in certain circumstances, the presence of solvent can be desired, particularly for solubilizing the organometallic complex and/or to homogenize the reaction medium. The amount of solvent to be used in of case is not critical.

Among the solvents suitable for the implementation of the method according to the invention we can cite water; alcohols, such as methanol, ethanol, 1-propanol, 2-propanol, 2-méthyl-1-propanol, 1-butanol, 2-butanol, tert-butanol, 1-pentanol, 2-pentanol, 3-pentanol, 1-hexanol, 2-methyl-1-pentanol, 2-ethyl-1-butanol, 1-heptanol, 1-octanol, 2-octanol, 1-nonanol or 1-decanol; aliphatic or aromatic hydrocarbons, halogenated or not, such as n-hexane, n-heptane, isooctane, nonane, decane, methylene chloride, chloroform, benzene, chlorobenzene, toluene, o-xylene, m-xylene, p-xylene, ethylbenzene, cyclohexane or methylcyclohexane; ethers such as tetrahydrofuran, dioxane, tetrahydropyran, 1,2-dimethoxyethane, 1,2-diethoxyethane, diethylene glycol dimethyl ether or diethylene glycol diethyl ether; ketones such as acetone or methyl ethyl ketone; sulfoxides such as dimethylsulfoxide; amides such as dimethylformamide or N-methylpyrrolidone; nitriles such as acetonitrile; or aromatic amines such as pyridine. The solvents particularly suitable for the implementation of the method according to the invention are selected from polar solvents having a low boiling point such as tetrahydrofuran, acetone, or methyl ethyl ketone. Indeed, it has been shown that this particular type of solvent was allowing homogenization of the reaction medium and promotoing of the condensation reaction.

Of course mixtures of multiple solvents can be used.

By means of the combination of an organometallic catalyst and a co-catalyst, the present invention offers the possibility to prepare compounds comprising at least one β-hydroxy-urethane unit and/or at least one γ-hydroxy-urethane unit in conditions of time and temperature acceptable industrially, preferably below 50° C., preferentially at ambiant temperature (approximately 25° C.).

Although it is possible to implement the method of the invention at a pressure above atmospheric pressure, it is most often preferred to operate at atmospheric pressure.

The duration of the condensation reaction depends on the temperature and nature of the condensing compound A with compound B as well as the catalyst employed. It can vary between 0.01 and 600 minutes, in particular, the reaction time is greater than 1 minute and not more than 480 minutes. Monitoring the progress of the reaction by sampling and evaluation of the formed product, by the technique of Fourier Transformation infrared spectroscopy (FTIR), enables a person of skill in the art to readily determine the reaction time and the most appropriate conditions.

The method according to the invention can be conducted under magnetic or mechanical agitation. It can be implemented in any reactor or equipment enabling creation of the conditions described above.

The method according to the invention can be carried out either continuously or batchwise. Industrially, the continuous method is preferred.

Compound A and compound B can be contacted with the catalyst comprising the organometallic complex and optionally the cocatalyst in various ways known per se.

A particularly preferred implementation mode of the invention is to make the first mixture of compound A and compound B, and then successively introduce the organometallic complex and the cocatalyst into the mixture under mechanical agitation at ambiant temperature and atmospheric pressure, and finally to add the solvent to homogenize the reaction mixture thus obtained.

At the end of the reaction, the reaction medium can be subjected to various known techniques for separation or purification, such as evaporation of the solvent (and optionally the co-catalyst) and drying in vacuo.

The high crude yields obtained under these conditions facilitate the production of compounds comprising at least one β-hydroxy-urethane unit and/or at least one γ-hydroxy-urethane high quality unit.

The present invention also relates to the use of a catalyst comprising at least one organometallic complex and a cocatalyst selected from the group of Lewis bases and/or salts of tetra-alkyl ammonium, to catalyze the condensation reaction between a compound A comprising at least one reactive unit and a cyclocarbonate compound B comprising at least amino reactive unit (—NH₂). The organometallic complex applied in such use contains an alkali, alkaline earth, or transition metal selected from Groups IA, IIA, IIIA, IIIB, IVA, IVB, VB, VIIB, VIIB and VIII of the periodic table, and at least two acetylacetonato ligands.

The method of the present invention therefore enables the preparation of compounds comprising at least one β-hydroxy-urethane unit and/or at least one γ-hydroxy-urethane unit of high quality, not having the disadvantages associated with urethanes prepared by the methods known in the prior art, that is to say, tackiness of resins prepared from hydroxyurethanes made by non-cyclocarbonate quantitative methods or disorders caused by the residual isocyanate-based materials in conventional polyurethanes.

Thus, the method of the present invention enables consideration of an easier, safer, and therefore greater use, of hydroxyurethanes, in particular compounds comprising at least one β-hydroxy-urethane unit and/or at least one γ-hydroxy-urethane unit, particularly in the fields of coatings, casting resins, flexible varnishes, impregnating resins for electrical insulation or alternatives, but also in the cosmetic, pharmaceutical or biomedical fields, or as intermediates for the preparation of crosslinked resins.

In the context of the preparation of hydroxyurethanes (linear or branched oligomers or linear or branched polymers) according to the method of the present invention, the mixture formed from compound A and compound B can further comprise a plasticizer (inert) before contacting with the catalyst comprising the organometallic complex and optionally the cocatalyst. The plasticizer (inert) operable to lower the viscosity of the compound A/compound B mixture and that of the final hydroxyurethane product, to improve the processability of the latter, particularly to facilitate the automated casting application. As examples of plasticizers that can be suitable for the preparation of hydroxyurethanes (linear or branched oligomers or linear or branched polymers) according to the method of the present invention, the plasticizers disclosed in patent U.S. Pat. No. 5,908,894 (Genz et al.) can be cited. Preferably, the plasticizer is selected from phthalic acid esters such as benzyl butyl phthalate (BBP), dibutyl phthalate (DBP), diethyl phthalate (DEP), dioctyl phthalate (DOP) or, di-2-ethylhexyl phthalate (DEHP); esters of succinic acid; adipic acid esters such as dibutyl adipate and dioctyl adipate; citric acid esters such as 2-(acetyloxy)-1,2,3-propanetricarboxylate tributyl; the isosorbide esters such as ID 37 Polysorb® EXP sold by the company Roquette Frères SA®; sulfonamides such as toluene sulfonamide marketed by the company Axcentive ® SA; phosphate esters or alkylsulfonic acid esters. Also preferentially, the plasticizer is selected from plasticizers derived from renewable resources such as diesters of isosorbide.

The amount of plasticizer applied can vary from 1 to 1000 moles of plasticizer per mole of compound A.

In a preferred implementation mode, the plasticizer is introduced into the reactor before the introduction of compound A and compound B.

From the description that has just been made of multiple variants of the method of the invention can be conceived by the person of skill in the art without departing from the scope of the invention defined by the claims.

The present invention is also described using the following examples, given by way of illustration without however limiting the scope.

### EXAMPLES

### Example 1

### Comparative Study of the Catalytic Activity of Catalysts Used in the Reaction Between a Cyclocarbonate Compound and a Diamine

In this example, the catalytic activity of the catalyst comprising tris(2,4-pentanedione) chromium (III) (SACHEM Europe BV), and optionally combined with triethylamine, was compared to the catalysts suggested in the known methods of the prior art catalysts, namely betaine (Sigma), tetraethylammonium hydroxide (Aldrich), 2-hydroxypyridine (Aldrich), tetraethylammonium tetrafluoroborate (Aldrich), tetramethylammonium bromide (Aldrich), triphenylphosphine (Aldrich), triethylamine (Sigma Aldrich), calcium carbonate (Aldrich). This study was carried out on the condensation reaction between trimethyl diamine (TMD Vestamin, Evonik) and 4-(hydroxymethyl)-1,3-dioxolan-2-one (Huntsman). The series of experiments carried out is explained in Table 1.

TABLE 1
Experimental conditions of the study of various catalytic activities of
the catalysts tested in the condensation reaction between trimethyl-
hexamethylene diamine and 4-(hydroxymethyl)-1,3-dioxolan-2-one.

| Quantity used of | Quantity used of | |
|---|---|---|
| 4-(hydroxymethyl)- | Trimethylhexameth- | Nature of the catalyst |
| 1,3-dioxolan-2-one | ylene diamine | [quantity used ] |
| 3.00 g (25.4 mmol) | 4.01 g (25.4 mmol) | Absence |
| 3.00 g (25.4 mmol) | 4.01 g (25.4 mmol) | Betaine [3.84 mg; |
| | | 0.19 mmol] |
| 3.00 g (25.4 mmol) | 4.01 g (25.4 mmol) | Tetraethylammonium |
| | | hydroxide [27.90 mg; |
| | | 0.19 mmol] |
| 3.00 g (25.4 mmol) | 4.01 g (25.4 mmol) | 2-Hydroxypyridine |
| | | [18.00 mg; 0.19 mmol] |
| 3.00 g (25.4 mmol) | 4.01 g (25.4 mmol) | Tetraethylammonium |
| | | tetrafluoroborate |
| | | [41, 20 mg; 0.19] |
| 3.00 g (25.4 mmol) | 4.01 g (25.4 mmol) | Calcium carbonate |
| | | [19,00 mg; 0.19 mmol] |
| 3.00 g (25.4 mmol) | 4.01 g (25.4 mmol) | Tetramethylammonium |
| | | bromide [29.20 mg; |
| | | 0.19 mmol] |
| 3.00 g (25.4 mmol) | 4.01 g (25.4 mmol) | Triphenylphosphine |
| | | [49.80 mg; 0.19 mmol] |
| 3.00 g (25.4 mmol) | 4.01 g (25.4 mmol) | Triethylamine |
| | | [19.20 mg; 0.19 mmol] |
| 3.00 g (25.4 mmol) | 4.01 g (25.4 mmol) | Tris(2,4-pentanedione) |
| | | chromium III |
| | | [40.53 mg; 0.12 mmol] |
| 3.00 g (25.4 mmol) | 4.01 g (25.4 mmol) | Tris(2,4-pentanedione) |
| | | chromium III] |
| | | [12.80 mg; 0.04 mmol] |
| | | and Triethylamine |
| | | [3.84 mg; 0.04 mmol] |

The conversion of 4-(hydroxymethyl)-1,3-dioxolan-2-one by the corresponding hydroxyuréthane was monitored by FTIR spectroscopy with a Nicolet 510P apparatus (FIG. 1).

In order to avoid the variations of band intensities between different analyses, an approximation of the conversion (a) of these functions has been defined from the area ratio (r). It is the ratio between the area of the characteristic band of carbonyl group belonging to the heterocycle 1,3-dioxolan-2-one located at 1800 cm⁻¹ and one centered at 769 cm⁻¹ assigned to Cₛₚ₂-H oxygenated heterocycle (FIG. 1):

$$r ( t ) = \frac{Aire vc = 0 ( t )}{Aire vcsp 2 - H ( t )}$$

The conversion into 4-(hydroxymethyl)-1,3-dioxolan-2-one a (t) over time is deduced:

$$a ( t ) = \frac{r ( o ) - r ( t )}{r ( o )}$$

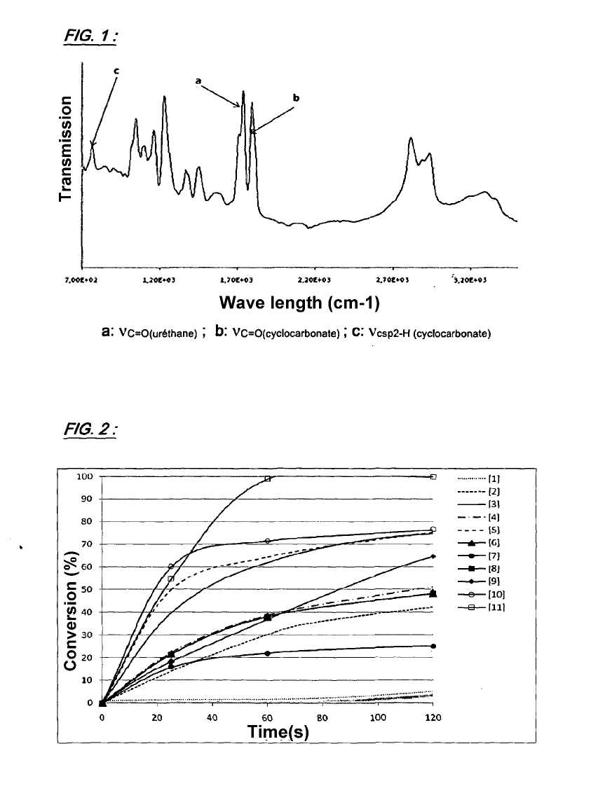

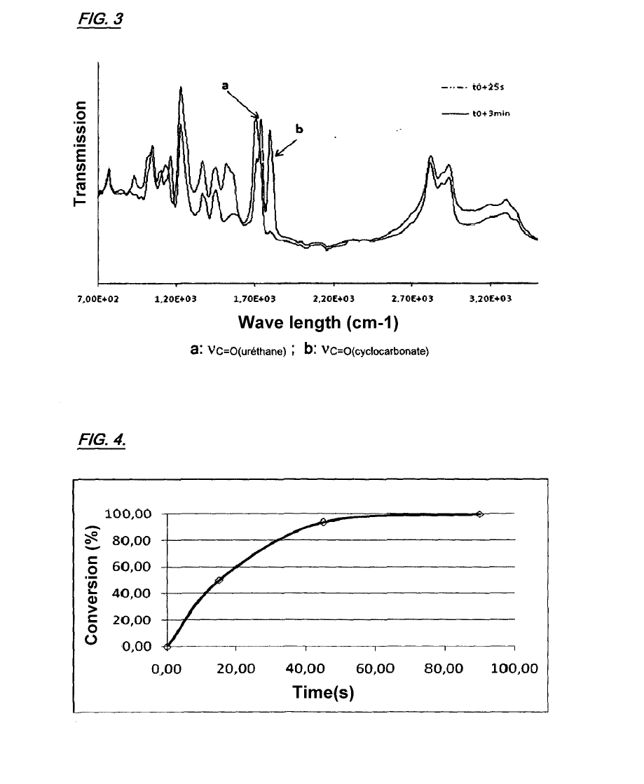

The results obtained are shown in FIG. 2 wherein the numbers in brackets correspond to the catalysts used: [1] betaine [2], tetraethylammonium tetrafluoroborate, [3] tetraethylammonium hydroxide, [4] 2-hydroxypyridine, [6] calcium carbonate [7] triphenylphosphine, [8] tetramethylammonium bromide, [9] triethylamine, [10] tris(2,4-pentanedione)chromium (III), [11] the catalyst comprising tris(2,4-pentanedione) chromium (III) and triethylamine.

From the data presented in FIG. 2, it appears that the use of tris(2,4-pentanedione) chromium (III) optionally added triethylamine enables an acceleration of the condensation reaction between 4-(hydroxymethyl)-1,3-dioxolan-2-one and diamine much higher than accelerations induced by the uses of catalysts previously described in the technical proposals. This catalytic system enables attainment of a quantitative conversion of the cyclocarbonate reactant in a very short time interval on the order of 120 seconds.

### Example 2

### Catalysis by the System tris(2,4-pentanedione) chromium (III)/triethylamine with Addition of trimethylhexamethylenediamine on (2-oxo-1,3-dioxolan-4-yl)methyl acetate

### 1. Preparation of (2-oxo-1,3-dioxolan-4-yl)methyl acetate

In a two-necked 250 ml flask fitted with a condenser, are introduced respectively: 4-(hydroxymethyl)-1,3-dioxolan-2-one (29.00 g, 0.22 mol), acetic anhydride (22.20 g, 0.21 mol, Sigma, Aldrich) and 2 drops of pure sulfuric acid. The reaction mixture, placed under mechanical agitation, is heated to 60° C., in air and at atmospheric pressure for 45 minutes.

The crude reactive is taken up in 80 ml of chloroform. The organic phase is washed ten times with 200 ml of distilled water, then dried over anhydrous magnesium sulfate.

The chloroform is evaporated in vacuo. The product is concentrated in a rotary evaporator under vacuum at 40° C. 20 g of a yellow low viscosity oil are obtained (yield=39.2%).

RMN¹H(DMSO): δ=2.5 ppm (3H, C₄H), 4.65 ppm (1H, C₁H), 4.70 ppm (1H, C₃H), 4.77 ppm (1H, C₃H), 5.12 ppm (1H, C₁H), 5.5 ppm (1H, C₂H). The RMN¹H spectrum was recorded with a 400 MHz Bruker AC 400 instrument.

### 2. Addition of the trimethylhexamethylenediamine to (2-oxo-1,3-dioxolan-4-yl) methyl acetate in the presence of tris(2,4-pentanedione)chromium (III)/triethylamine

In a test tube were introduced in the absence of solvent, (2-oxo-1,3-dioxolan-4-yl)methyl acetate (4.06 g, 25.40 mmol), trimethylhexamethylenediamine (4, 01 g, 25.40 mmol), tris(2,4-pentanedione) chromium (III) (12.80 mg, 0.04 mmol) and triethylamine (3.84 mg, 0.04 mmol).

The reaction mixture placed under mechanical agitation is left at ambiant temperature in air and at atmospheric pressure. The consumption of (2-oxo-1,3-dioxolan-4-yl)methyl acetate after mixing of the reagents was determined by FTIR spectroscopy (FIG. 3) following the protocol described in Example 1.

The study of the conversion of the (2-oxo-1,3-dioxolan-4-yl) methyl acetate group in the corresponding hydroxyurethane conducted by infrared spectroscopy shows quantitative conversion in less than 3 minutes ambient temperature of this reagent. Therefore, use of the catalytic system consisting of tris(2,4-pentanedione) chromium (III) of triethylamine additive remains very efficient to accelerate the condensation reaction between the diamine and trimethylhexamethylene (2-oxo-1,3-dioxolan-4-yl) methyl acetate.

### Example 3

### Catalysis by the system tris(2,4-pentanedione) chromium (III)/triethylamine addition of tetraethylene pentamine on 4-(hydroxymethyl)-1,3-dioxolan-2-one

In a test tube were introduced in the absence of solvent, the 4-(hydroxymethyl)-1,3-dioxolan-2-one (3.00 g, 25.4 mmol), tetraethylene pentamine (5.10 g, 25, 4 mmol, Huntsman), tris(2,4-pentanedione) chromium (III) (12.80 mg, 0.04 mmol) and triethylamine (3.84 mg, 0.04 mmol). The reaction mixture, placed under mechanical agitation, is left at ambiant temperature in air and at atmospheric pressure.

The conversion of 4-(hydroxymethyl)-1,3-dioxolan-2-one corresponding hydroxyuréthane was followed by FTIR, following the protocol described in Example 1. The results obtained are shown in FIG. 4.

From the data presented in FIG. 4, it is clear that the introduction of catalytic amounts of tris(2,4-pentanedione) chromium (III) and triethylamine enables attainment of a quantitative consumption at ambiant temperature of 4-(hydroxymethyl)-1,3-dioxolan-2-one in 60 seconds after mixing of tetraethylenepentamine with 4-(hydroxymethyl)-, 3-dioxolan-2-one.

### Example 4

### Catalysis by the system tris(2,4-pentanedione) chromium (III)/triethylene amine of the condensation reaction between the 3-aminomethyl-3,5,5-trimethylcyclohexylamine and 4-(hydroxymethyl)-1,3-dioxolan-2-one

In a test tube were introduced in the absence of solvent, 4-(hydroxymethyl)-1,3-dioxolan-2-one (3 g, 25.4 mmol), 3-aminomethyl-3,5,5-trimethylcyclohexylamine (4.32 g, 25.4 mmol, Evonik Vestamin IPD), tris(2,4-pentanedione) chromium (III) (12.8 mg, 0.04 mmol) and triethylamine (3.84 mg, 0, 04 mmol). The reaction mixture placed under mechanical agitation is left at ambiant temperature in air and at atmospheric pressure. The study of the progress of the reaction is performed by FTIR, following the protocol described above.

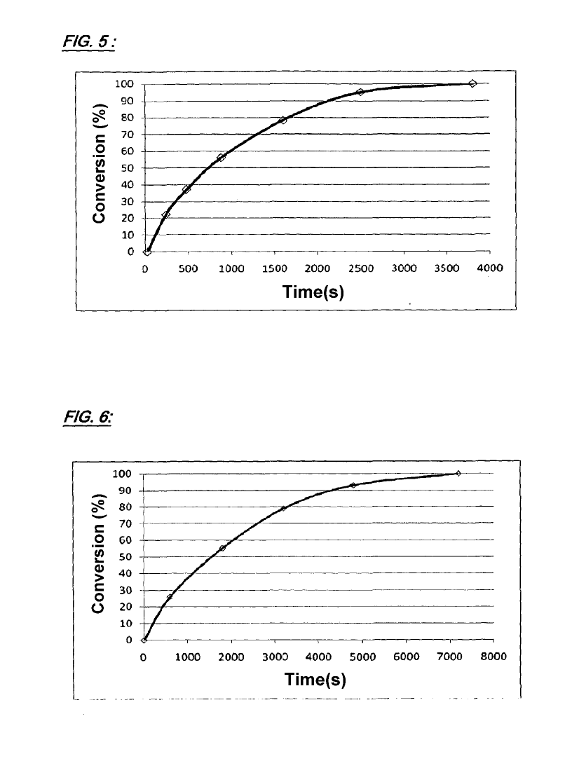

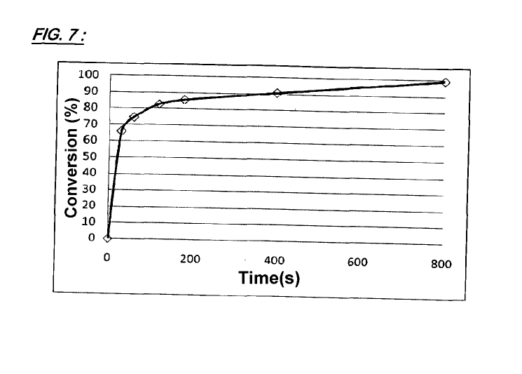

The study of the conversion, of 4-(hydroxymethyl)-1,3-dioxolan-2-one into the corresponding hydroxyuréthane, by FTIR spectroscopy (FIG. 5) during the addition reaction carried out at ambiant temperature, highlights that that this catalyst system enables quantative consumption at ambiant temperature in of the reactive functions in a time interval of approximately an hour after mixing the reagents.

### Example 5

### Catalysis by the system tris(2,4-pentanedione) chromium (III)/triethylamine addition of Jeffamine D230 on 4-(hydroxymethyl)-1,3-dioxolan-2-one

In a test tube were introduced in the absence of solvent, the 4-(hydroxymethyl)-1,3-dioxolan-2-one (3.0 g, 25.4 mmol), Jeffamine D230 (5.84 g, 25, 4 mmol, Huntsman Jeffamine D230), tris(2,4-pentanedione) chromium (III) (12.80 mg, 0.04 mmol, SACHEM Europe BV), and triethylamine (3.84 mg, 0.04 mmol, Sigma Aldrich). The reactive mixture, placed under mechanical agitation, is left at ambiant temperature in air and at atmospheric pressure. Monitoring the progress of the reaction is achieved by FTIR spectroscopy, following the protocol described above.

From the data presented in FIG. 6, it is observed that the introduction of catalytic amounts of tris(2,4-pentanedione) chromium (III)/triethylamine additive enables attainment of a quantitative consumption, at ambiant temperature, of the cylocarbonaté reactant in a time interval of 2 hours after mixing of Jeffamine D230 with 4-(hydroxymethyl)-1,3-dioxolan-2-one.

### Example 6

### Addition of a diamine tetraethylenepentamine bis-cyclocarbonate catalyzed by the system tris(2,4-pentanedione) chromium (III)/triethylene amine

### 1. Synthesis of bis-cyclocarbonate by addition of 4-(hydroxymethyl)-1,3-dioxolan-2-one on the Desmodur N3400

In a two-necked 250 mL flask previously dried and weighed are introduced respectively the diisocyanate prepolymer based on hexamethylene diisocyanate (0.26 moles, 100 g Desmodur N3400 Bayer), 4-(hydroxymethyl)-1,3-dioxolan-2-one (0.53 mol, 61, 4 g) and dioctyl tin dilaurate (0.001 moles, 0.744 g, Thorson Chemical GmbH). The reaction mixture is left under magnetic agitation at ambiant temperature and under atmospheric pressure for 2 h.

The crude reaction mixture is dissolved in 100 ml of tetrahydrofuran and precipitated in 1 L of distilled water. This procedure is repeated a second time. The precipitate is dissolved in tetrahydrofuran (100 mL) then dried over anhydrous magnesium sulfate. The product is concentrated in a rotary evaporator under vacuum at 40° C. 150 g of a highly viscous oil of white color are obtained (yield=93.0%).

The FTIR analysis of the obtained product shows the absence of the focus-band at 2250 cm⁻¹ characteristic of isocyanate functional groups (—NCO) of Desmodur N3400 and the presence in 1783 cm⁻¹ band of the carbonyl (C═O) of cyclocarbonate.

### 2. Catalysis system tris(2,4-pentanedione) chromium (III)/triethylamine polyaddition of tetraethylene the bis-cyclocarbonate derived from Desmodur N3400

In a glass beaker are introduced: the bis-cyclocarbonate derivative of Desmodur N3400 (16.46 g, 25.4 mmol), tetraethylene pentamine (5.10 g, 25.4 mmol, Huntsman), tris(2,4-pentanedione) chromium (III) (12.8 mg, 0.04 mmol, SACHEM Europe BV) and triethylamine (3.84 mg, 0.04 mmol, Sigma Aldrich). The reaction medium is homogenized by adding 2.0 ml of tetrahydrofuran.

### Example 7

### Study of catalyzing the condensation reaction between the 3-aminomethyl-3,5,5-trimethylcyclohexylamine and glycerol carbonate by various catalysts

An operating mode similar to that of the preceding examples is used: same conditions of temperature and pressure.

The catalysts (organometallic complex/cocatalyst) tested are as follows: [11] tris(acetylacetonate) chromium (III)/triethylamine; [12] tris(acetylacetonate) aluminum (III)/triethylamine; [13] bis(acetylacetonate) calcium (II); [14] bis(acetylacetonate) calcium (II)/tetrabutylammonium bromide; [15] bis(acetylacetonate) calcium (II)/triethylamine; [16] bis(acetylacetonate) cobalt (II)/triethylamine; [17] bis(acetylacetonate) nickel (II)/triethylamine; [18] tris(acetylacetonate) iron (III)/triethylamine; [19] bis(acetylacetonate) zinc (II)/triéthyalmine.

The following organometallic complexes: tris(acetylacetonate) chromium (III), tris(acetylacetonate) aluminum (III); bis(acetylacetonate) cobalt (II); bis(acetylacetonate) nickel (II); tris(acetylacetonate) iron (III); bis(acetylacetonate) zinc (II); bis(acetylacetonate) calcium (II) are available from SICCANOR®. Tetrabutylammonium bromide is available from Sigma Aldrich®.

For each test, we used: 10 g (58.8 mmol) of 3-aminomethyl-3,5,5-trimethylcyclohexylamine (isophorone diamine) and 6.9 g (58.4 mmol) of 4-(hydroxymethyl)-1,3-dioxolan-2-one and the catalyst system tested “0.06 mmol of the organometallic complex in combination with 0.06 mmol cocatalyst”.

These tests showed that the catalysts used enable different reaction times shorter than 5 minutes at ambiant temperature. The best results were obtained with the organometallic complex bis(acetylacetonate) calcium (II) in combination with the co-catalyst triethylamine. Indeed, it was found a time less than 30 seconds of reaction with the catalyst bis(acetylacetonate) calcium (II) (0.06 mmol)/triéthyalmine (0.12 mmol) at ambiant temperature.

### Example 8

### The condensation reaction between 2,2,4-trimethylhexamethylene diamine and a mixture composed of tricyclocarbonate (Laprolat ETF) and a dicyclocarbonate (Laprolat E-181) in the presence of bis(pentane-2,4-dionoto)calcium)/triethylamine.

Laprolat E-181 is a dicyclocarbonate resin prepared from di-epoxy oligoethers and marketed by MACROMER Ltd®. It has a molecular weight of 400 g/mol, the weight percentage in cylocarbonate representing 44.5% by weight.

Laprolat ETF is a resin of the type tricylocarbonate (oligoether-oxyphenyl) methane, which is marketed by the company MACROMER Ltd®. It has a molecular weight MW=750 g/mol, the mass percentage cylocarbonate representing 44.5% by weight.

In a beaker was introduced Laprolat E-181 (16.3 g, 40.75 mmol) and Laprolat ETF (4.0 g, 5.33 mmol). The whole is heated at 90° C. for 4 h to obtain a homogeneous liquid mixture. To the mixture, returned to ambiant temperature, are added, with agitation, bis(pentane-2,4-dionato) calcium (200 mg, 0.84 mmol, Siccanor), triethylamine (84.9 mg, 0.84 mmol, Sigma Aldrich®), trimethylhexamethylene diamine (7.7 g, 48.6 mmol), Vestamin® TMD Evonik Degussa GmbH). Then, changes in the rigidity of the reaction medium during the time were made at ambiant temperature by means of a TROMBOMAT® device (marketed by PRODEMAT® SA) which is able to determine the transistion to the gel point.

A very short gel time, less than 1 minute, was observed.

### Example 9

### Preparation of a hybrid epoxy/hydroxylated polyurethane

Into a beaker was introduced at atmospheric pressure, at ambiant temperature, and under mechanical agitation, propylene carbonate (0.8 g, 7.8 mmol, Huntsman®), glycerol carbonate (3.0 g, 25.4 mmol Huntsman®), Laprolat® ETF (2.0 g, 2.7 mmol, MACROMER®), triépoxy YH® 300 (12.5 g, EEW=142.5 g/eq, Kukdo ®), bis(acetylacetonate) calcium (II) (200 mg, 0.84 mmol, Siccanor®), triethylamine (54.5 mg, 0.55 mmol Sigma Aldrich®), Dolomite DRB 3® (3.6 g, calcium oxide dual magnesium, Imerys Performance Minerals®). The charged mixture is stirred 1 h at ambiant temperature.

Then trimethyl-hexamethylenediamine “Vestamine TMD®” (3.26 g, 20.63 mmol, Evonik Degussa GmbH) was added. The mixture is left at ambiant temperature for 1 h, then heated 30 minutes at 100° C. to finish the polymerization/crosslinking and give the material its final mechanical properties.

Dolomite DRB 3 was used as a mineral filler.

The polyhydroxyuréthane hybrid thus obtained exhibits the following properties:
At 23° C. Hardness (Shore 70 A, according to standard ISO 868);
Maximum Strength at break 160N (Standard ISO R 527);
Maximum Strain at break 38.22% (standard ISO R 527)
Young's modulus: 8.27 MPa,
Water Reprise performed by immersion in boiling water 1 h 2.2%.

1. A method for preparing a compound comprising a β-hydroxy urethane unit or a γ-hydroxy-urethane unit, comprising reacting a compound A comprising a cyclocarbonate reactive unit with a compound B comprising an amino reactive unit (—NH₂) in the presence of a catalyst, said method being characterized in that said catalyst comprises an organometallic complex and a cocatalyst selected from the group of Lewis bases, or salts of tetra-alkyl ammonium.

2. A method according to claim 1, characterized in that the organometallic complex contains an alkali alkaline earth, or transition metal selected from groups IA, IIA, IIIA, IIIB, IVA, IVB, VB, VIIB, VIIB and VIII of the periodic table, and two β-diketonate ligands.

3. A method according to claim 2, characterized in that the β-diketonate ligands are selected from the following list: acetylacetonato, 1,1,1,5,5,5-hexa-fluoro-acetyl, 1,1,1-trifluoro-acetylacetonate, 1,1,1-trifluoro-5,5-di-méthylacétylacétonate and 2,2,6,6-tetramethyl-3,5-heptanedionate.

4. A method according to claim 1, characterized in that the organometallic complex is selected from the following list: tris(acetylacetonate) aluminum(III), tris(hexafluoroacetylacetonate) aluminum(III), tris(trifluoroacetylacetonate) aluminum(III), tris(2,2,6,6-tetramethyl-3,5-heptanedionato) aluminum(III), bis(acetylacetonate) calcium(II), tris(acetylacetonate) chromium(III), tris(2,2,6,6-tetramethyl-3,5-heptanedionate) chromium(III), tris(acetylacetonate) cobalt(III), tris(acetylacetonate nitro) cobalt(III), tris(2,2,6,6-tetramethyl-3,5-heptanedionate) cobalt(III), bis(acetylacetonate) copper(III), bis(2,2,6,6-tetramethyl-3,5-heptanedionate) copper(III), tris(acetylacetonato) gallium (III), acetylacetonate hafnium(IV), tris(acetylacetonato) indium(III), tris(acetylacetonate) iron(III), tris(2,2,6,6-tetramethyl-3,5-heptanedionate) iron(III), tris(acetylacetonate) manganese(III), bis(acetylacetonate) nickel(II), bis(acetylacetonate) palladium (II), bis(trifluoroacetylacetonate) palladium (II), acetylacetonate sodium bis(acetylacetonate) titanium oxide(IV), bis(2,2,6,6-tetramethyl-3,5-heptanedionate) titanium oxide(IV), tris(2,2,6,6-tetramethyl-3,5-heptanedionato)-titanium(III), tris(2,2,6,6-tetramethyl-3,5-heptanedionato)oxotitanium(IV), dichloro-bis(2,2,6,6-tetramethyl-3,5-heptanedionato) titanium(IV), (diisopropoxide)bis(acetylacetonate) titanium(IV), di(isopropoxide)bis(2,2,6,6-tetramethyl-3,5-heptanedionato) titanium(IV), bis(acetylacetonate) zinc (II), tetrakis(acetylacetonate) zirconium(IV), tetrakis(hexafluoroacetylacetonate) zirconium(IV), tetrakis(2,2,6,6-tetramethyl-3,5-heptanedionato) zirconium(IV), tetrakis(trifluoroacetylacetonate) zirconium(IV).

5. A method according to claim 1, characterized in that the molar organometallic complex/compound A ratio is from 0.001 to 0.05.

6. A method according to claim 1, characterized in that the cocatalyst is selected from trialkyl amines, aromatic heterocyclic amines, trialkylphosphines, triarylphosphines, trialkyl phosphites, triaryl phosphites, tetralakyl ammonium salts, or mixtures thereof.

7. A method according to claim 1, characterized in that the cocatalyst is used at a porportion of 1.5 to 3 moles per mole of organometallic complex.

8. A method according to claim 1, characterized in that compound A is selected from the group of compounds, oligomers or polymers, terminated with a cyclocarbonate.

9. A method according to claim 1, characterized in that compound A is a compound satisfying:
the general formula A1: 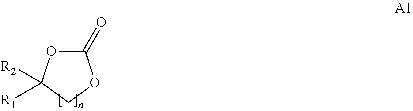
wherein:
n represents an integer of 1 or 2,
R₁ représente un atome d′hydrogéne ou un groupe alkyle en C₁₋₆, linéaire ou ramifié, saturé ou insaturé, et le cas échéant, substitué par un ou plusieurs substituants choisis parmi halogéne, hydroxy, cyano, carboxy, trifluorométhyl, alkyl en C₁₋₆, alkylamino en C₁₋₆, dialkylamino en C₁₋₆, alcoxy en C₁₋₆, alkylcarboxylate en C₁₋₆,] R₁ represents a hydrogen atom or a C₁₋₆ alkyl group, linear or branched, saturated or unsaturated,
R₂ represents a hydrogen atom or a group selected from: a C₁₋₂₀ alkyl, linear or branched, saturated or unsaturated, and wherein non-neighboring methylene units can be replaced by radicals —O—, —S—, —S(O)—, —SO₂—, —O—C(═O)—, —N(R²¹)—, —N(R²¹)—C(═O)—, —N(R²¹)—C(═O)—O—, —N(R²¹)—C(═O)—N(R²²)—, with R²¹ and R²² being identical or different, representing hydrogen or selected from C₁₋₆ alkyl or aryl of C₆₋₁₄ carbon radicals; C₃₋₁₀ cycloalkyl; C₆₋₁₄ aryl; C₃₋₁₀ heterocycle having a heteroatom selected from N, O, S; C₄₋₁₃ heteroaryl having a heteroatom selected from N, O, S
R₅ represents:
a direct bond,
a bivalent group selected from: a C₁₋₂₀ alkylene, linear or branched, saturated or unsaturated, and wherein non-neighboring methylene units may be replaced by radicals —O—, —S—, —S(O)—, —SO₂—, —C(═O)—O—, —N(R²¹)—, —N(R²¹)—C(═O)—, —N(R²¹)—C(═O)—O—, —N(R²¹)—C(═O)—N(R²²)—, with R²¹ and R²² are identical or different, representing hydrogen or selected from C₁₋₆ alkyl or C₆₋₁₄ arly radicals; a C₃₋₁₀ cycloalkylene; a C₆₋₁₄ arylene; C₃₋₁₀ heterocyclene having a heteroatom selected from N, O, S; C₄₋₁₃ heteroarylene having a heteroatom selected from N, O, S
or a bivalent group having the following general formula I: 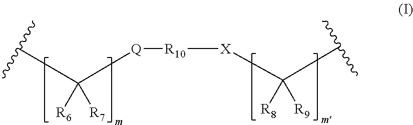
wherein
m and m′, identical or different, designate an integer from 1 to 100,
R₆, R₇, R₈ and R₉, identical or different, represent hydrogen atoms or C₁₋₆ alkyl, linear or branched, saturated or unsaturated,
Q and X, identical or different, represent a group —O—C(═O)—, —N(R²¹)—, —N(R²¹)—C(═O)—, —N(R²¹)—C(═O)—O—, —N(R²¹)—C(═O)—N(R²²)—, with R²¹ and R²² as defined above,
R¹⁰ represents a bivalent group selected from: a C₁₋₂₀ alkylene, linear or branched, saturated or unsaturated, and wherein non-neighboring methylene units can be replaced by radicals —O—, —S—, —S(O)—, —SO₂—, —C(═O)—O—, —N(R²¹)—, —N(R²¹)—C(═O)—, —N(R²¹)—C(═O)—O—, —N(R²¹)—C(═O)—N(R²²)—, with R²¹ and R²², identical or different, representing hydrogen or groups selected from C₁₋₆ alkyl or C₆₋₁₄ aryl; a C₃₋₁₀ cycloalkylene; C₆₋₁₄ arylene; C₃₋₁₀ heterocyclene having a heteroatom selected from N, O, S; C₄₋₁₃ heteroarylene having a heteroatom selected from N, O, S; the members of this group being bivalent;
or, a group satisfying one of the formulas II or III:
—(CH₂—CH₂—O)ₜ—CH₂—CH₂— (II), or
—C(═O)—CH₂—O—(CH₂—CH₂—O)_{t′}—CH₂—C(═O)— (III)
wherein:
t, t′, identical ou different, designate a whole number from 1 to 100.

10. A method according to claim 1, characterized in that compound A is a compound A3 obtained by:
reacting 4-hydroxymethyl-1,3-dioxolan-2-one with a compound comprising two isocyanates functions
the esterification reaction of 4-hydroxymethyl-1,3-dioxolan-2-one with a compound comprising a carboxylic acid function,
the reaction of triglycidyl isocyanurate with carbon dioxide, or
the reaction of a compound comprising two glycidyl ether groups with carbon dioxide.

11. A method according to claim 1, characterized in that compound B is selected from the group of compounds, oligomers or polymers terminated with a reactive unit (—NH₂).

12. A method according to claim 1, characterized in that the reactive units (—NH₂) are carried by saturated carbons (sp3), which are also carriers of a hydrogen.

13. A method according to claim 1, characterized in that compound B comprising an amino reactive unit (—NH₂) is selected from the following list: butylamine; hexylamine; cyclohexylamine; ethanolamine; propanolamine; ethylene diamine; propylene diamine; butylene diamine; pentamethylene diamine; hexamethylene diamine; heptamethylene diamine; tetramethylene diamine; octamethylene diamine; nonamethylene diamine; decamethylene diamine; 2-methyl-pentamethylene diamine; undecamethylene diamine; dodecamethylene diamine; xylylene diamine; isophorone diamine; trimethyl hexamethylene diamine; 1,2-diaminocyclohexane; 1,4-diaminocyclohexane; 4,4′-diaminocyclohexyl methane; 2-(2-aminoethoxy)ethanol; bis-(3-methyl-4-aminocyclohexyl) methane; 2,2-bis(4-aminocyclohexyl) propane; nitrile tri(éthaneamine); bis-(3-aminopropyl) methylamine; 3-amino-1-(methylamino) propane; 3-amino-1-(cyclohexylamino) propane; N-(2-hydroxyethyl)ethylene diamine; 4,7-dioxadecane-1,10-diamine; 4,9-dioxadodécane-1,12-diamine; 7-methyl 4,10-dioxamidécane-1,13-diamine; a polyether monoamine, a polyether diamine or an oligomer or polymer terminated by a amino reactive unit (—NH₂), said oligomer or polymer capable of having a polyamide, polyether or polyester skeleton.

14. A method according to claim 1, characterized in that compound A and compound B are mixed so that the molar ratio of compound B over compound A is between 0.5 and 2.

15. A method according to claim 1, characterized in that compound A and compound B are pre-mixed with a plasticizer before contacting with the catalyst, which catalyst comprises the organometallic complex and the cocatalyst.

16. Use of a catalyst comprising an organometallic complex and a cocatalyst selected from the group of Lewis bases or salts of tetra-alkyl ammonium, to catalyze the condensation reaction between a compound A comprising a cyclocarbonate reactive unit and a compound B comprising an amino reactive unit (—NH₂).
